(19) United States
(12) Patent Application Publication
(10) Pub. No.: US 2015/0086101 A1
(43) Pub. Date: Mar. 26, 2015

(54) METHOD FOR ORGAN LOCALIZATION

(71) Applicant: GENERAL ELECTRIC COMPANY, Schenectady, NY (US)

(72) Inventors: Roshni Bhagalia, Niskayuna, NY (US); Qi Song, Niskayuna, NY (US); Albert Amos Montillo, Niskayuna, NY (US)

(21) Appl. No.: 14/496,639

(22) Filed: Sep. 25, 2014

Related U.S. Application Data
(60) Provisional application No. 61/882,415, filed on Sep. 25, 2013.

(51) Int. Cl. A61B 6/00; G06K 9/46; G06T 7/00; A61B 6/03

(52) U.S. Cl. CPC A61B 6/5241; A61B 6/032; A61B 6/463; A61B 6/469; A61B 6/488; G06K 9/46; G06T 7/0012; G06T 2207/30004; G06K 2009/4666
USPC 382/131; 378/4

(57) ABSTRACT

In some embodiments, a method for localizing organs in anatomical imaging may include: performing an anterior-posterior view scan and a lateral view scan to create an anterior-posterior view scan image and a lateral view scan image; creating a joint anatomical model based on the anterior-posterior scan image and the lateral view scan image; and refining the joint anatomical model.

### CROSS-REFERENCE TO RELATED APPLICATION

This application claims the benefit of U.S. Provisional Application No. 61/882,415, filed Sep. 25, 2013.

### BACKGROUND

The subject matter disclosed herein generally relates to anatomical imaging, and more specifically, to localizing organs in anatomical imaging.

Conventional anatomical imaging utilizes one or more preliminary scans (e.g., scout, topogram, survey or the like) to define a region of interest and/or plot locations for slice images in a subsequent full scan. Typically, information provided from one of an anterior-posterior (AP) or lateral (LAT) view image is utilized to define a general region of interest for the subsequent scan. However, the inventors have observed that such techniques do not provide suitable accuracy, often requiring a longer scan and/or wider area of the patient's body to be scanned, thereby exposing the patent to a higher radiation dose.

Therefore, the inventors have provided an improved method for localizing organs in anatomical imaging.

### SUMMARY

Embodiments of method for localizing organs in anatomical imaging are provided herein.

In some embodiments, a method for localizing organs in anatomical imaging may include: performing an anterior-posterior scan and a lateral view scan to create an anterior-posterior scan image and a lateral view scan image; creating a joint anatomical model based on the anterior-posterior scan image and the lateral view scan image; and refining the joint anatomical model.

In some embodiments, a computer readable medium, having instructions stored thereon which, when executed, causes an imaging system to perform a method for localizing organs in anatomical imaging, wherein the method may include: performing an anterior-posterior scan and a lateral view scan to create an anterior-posterior scan image and a lateral view scan image; creating a joint anatomical model based on the anterior-posterior scan image and the lateral view scan image; and refining the joint anatomical model.

The foregoing and other features of embodiments of the present invention will be further understood with reference to the drawings and detailed description.

### DESCRIPTION OF THE FIGURES

Embodiments of the present invention, briefly summarized above and discussed in greater detail below, can be understood by reference to the illustrative embodiments of the invention depicted in the appended drawings. It is to be noted, however, that the appended drawings illustrate only typical embodiments of the invention and are therefore not to be considered limiting in scope, for the invention may admit to other equally effective embodiments.

FIG. 1 is a method for localizing organs in anatomical imaging, in accordance with some embodiments with the present invention.

FIGS. 2A-D depict anatomical based images which may be utilized in the method described in FIG. 1, in accordance with some embodiments of the present invention.

FIG. 3 depicts anatomical based images which may be utilized in the method described in FIG. 1, in accordance with some embodiments of the present invention.

FIG. 4 is a pictorial view of a computed tomography (CT) imaging system suitable for performing at least a portion of the inventive method, in accordance with some embodiments of the present invention.

FIG. 5 is a block schematic diagram of the system illustrated in FIG. 4.

To facilitate understanding, identical reference numbers have been used, where possible, to designate identical elements that are common to the figures. The figures are not drawn to scale and may be simplified for clarity. It is contemplated that elements and features of one embodiment may be beneficially incorporated in other embodiments without further recitation.

### DETAILED DESCRIPTION

Embodiments of method for localizing organs in anatomical imaging are provided herein. The inventive method advantageously utilizes complementary information provided by both anterior-posterior (AP) or lateral (LAT) view images and an integrated analysis of such information to provide an increased localization accuracy, thereby allowing for a shorter and more accurately targeted full dose scan and, thus reducing radiation dosing of a patient.

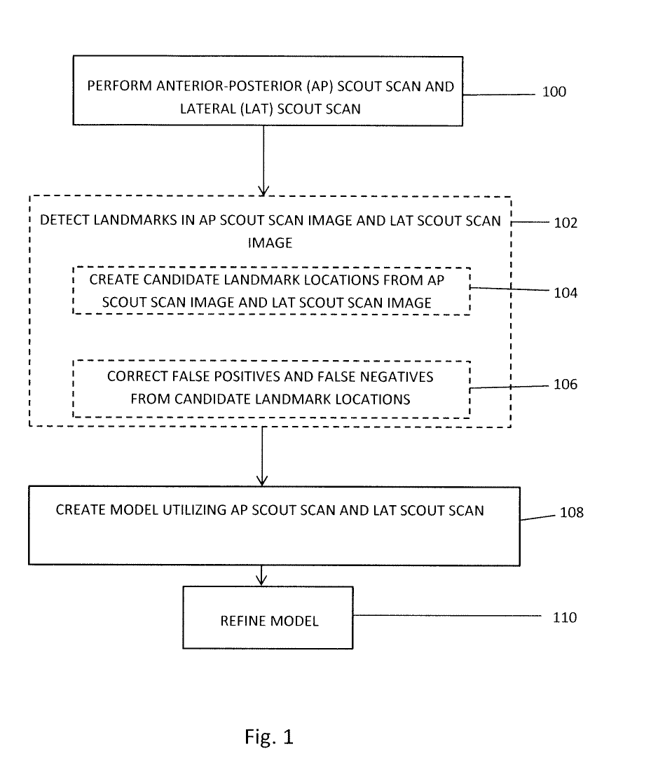

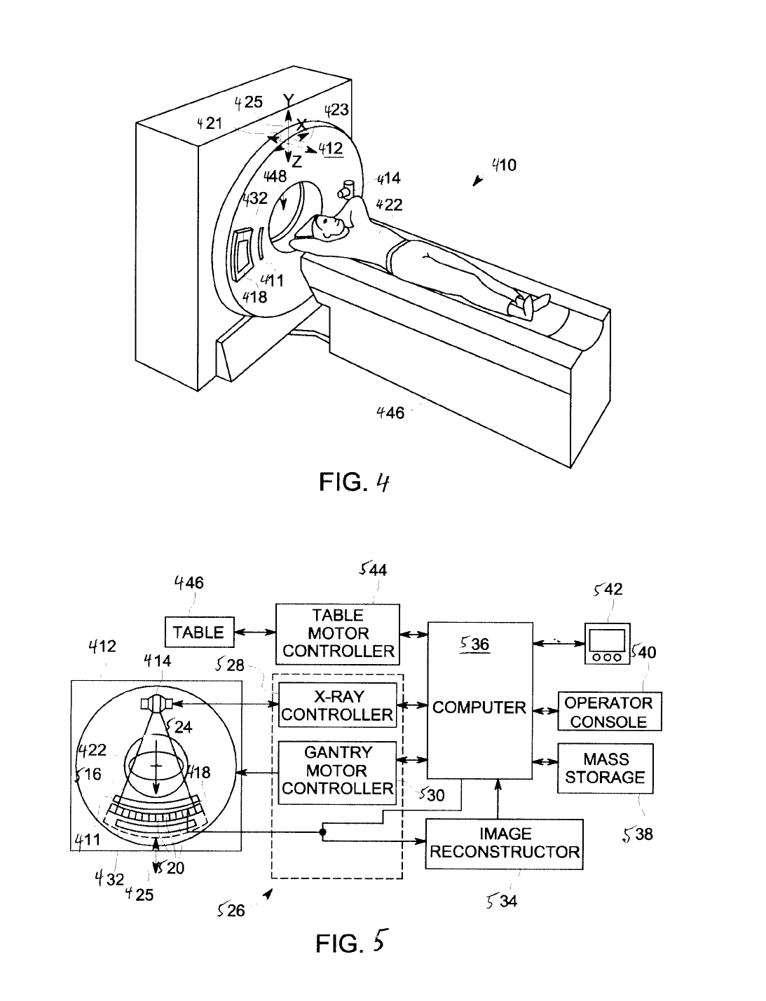

FIG. 1 is a flow diagram of the inventive method 100 for localizing organs in anatomical imaging, in accordance with some embodiments with the present invention. The method 100 may be performed utilizing any system suitable for anatomical imaging, for example, such as the exemplary CT system shown in FIGS. 4 and 5.

The method 100 generally starts at 110, where an anterior-posterior (AP) scout scan and a lateral (LAT) scout scan are performed. The AP scout scan and the LAT scout scan may be performed in any manner suitable to provide sufficient information for creating and refining the joint anatomical model, as described below. For example, in some embodiments, a general target region of a patient disposed within a CT system (e.g., such as the patient 422 disposed on the table 446 of the CT system 410 described below) is determined by an operator. X-rays are then delivered while a gantry is rotated to a fixed position and the table is moved with respect to the gantry (e.g., such as the gantry 412, x-rays 516 and table 446 described below). The x-rays are collimated, processed and an image constructed to provide the AP and LAT scout scan images (e.g., such as via the detector 520, data acquisition systems (DAS) 432 and collimator assembly 411 described below).

The inventors have observed that conventional imaging techniques typically utilize information provided from one of an anterior-posterior (AP) or lateral (LAT) view image is to define a general region of interest for the subsequent scan. However, the inventors have observed that such information is typically not sufficient to provide suitable accuracy for a subsequent targeted scan. Such lack of accuracy often results in an increased scan time and/or wider area of the patient's body needing to be scanned in the subsequent full dose scan, thereby exposing the patent to a higher radiation dose. As such, as will be described in further detail below, the inventors have observed that by utilizing complementary information provided by both the anterior-posterior (AP) or lateral (LAT) view image and performing an integrated analysis of such information an increased localization accuracy (as compared to conventionally performed techniques) may be achieved, thereby allowing for a shorter and more accurately targeted full dose scan and, thus reducing radiation dosing of a patient.

Next, and optionally at 102, one or more landmarks in both the AP scout scan image and the LAT scout scan image may be detected. The one or more landmarks may be detected via any suitable mechanism to provide initial locations of salient landmarks for both the AP and LAT scout scan images.

For example, to detect the one or more landmarks, first at 104, candidate landmark locations from the AP and LAT scout scan images may be created. The candidate landmark locations may be created via any technique suitable to provide sufficiently accurate candidate landmark locations. For example, in some embodiments, a rejection cascade classifier framework may be utilized to determine whether a particular landmark is present in each of the AP and LAT scout scan images. The rejection cascade may be built using a learning algorithm, for example, an adaptive boosting algorithm such as Gentle AdaBoost. Each cascade may be applied as a sliding window classifier to determine if and where a particular landmark is present in the AP and LAT scout scan images.

When utilized, in some embodiments, the rejection cascade classifier may be trained for each landmark via supervised learning. In such embodiments, each of the AP and LAT scout scan images may be annotated with landmarks manually. The manually annotated landmarks may include any landmarks suitable to provide accurate candidate landmark locations via the rejection cascade classifier. For example, in some embodiments, the manual landmarks of the AP scout scan image may include any or all of a heart-diaphragm intersection, lung corners, diaphragm peak, regions or locations on the lung, airway-lung intersections, regions or locations on the heart, regions or locations of the ribcage, or the like. In some embodiments, manual landmarks of the LAT scout scan image may include ends of the diaphragm, spine-diaphragm intersection, regions or locations on the lung, posterior of the spine, regions or locations on the lung, regions or locations on the heart, heart-diaphragm intersection, or the like. Following the manual annotation of the AP and LAT scout scan images, features may be identified and computed via cropping of the AP and LAT scout scan images and object recognition techniques (e.g., utilized Haar templates, or the like).

Next, at 106, false positives and false negatives from the candidate landmark locations may be corrected. The false positives and false negatives may be corrected via any technique suitable to accurately identify each of the false positives and false negatives. For example, in some embodiments, a generative model of the geographic configuration of the landmarks may be utilized to correct the false positives and false negatives of the AP and LAT scout scan images. In such embodiments, an expected location of a landmark learned from a previous set of trained images may be utilized to determine which candidate landmark location from each of the AP scout scan and the LAT scout scan is more accurate. For example, in instances where the AP scout scan and the LAT scout scan each provide a distinct location for a given landmark, a single candidate landmark from either the AP scout scan or the LAT scout scan having the lowest uncertainty estimated by its median Mahalanobis distance may be retained. In addition, in some embodiments, landmarks that are missing in the candidate landmark locations may be inferred based on estimated positions of the missing landmarks provided by previous sets of trained images.

Next, at 108, a model (joint anatomical model) is created utilizing the AP scout scan and the LAT scout scan. The model may be created using any information provided by both the AP scout scan and the LAT scout scan. For example, information provided by any rough segmentation or detection methods known in the art, or manual user input may be utilized to create the model. In one example, in some embodiments, the anterior-posterior view scan image and the lateral view scan image may be segmented to provide a plurality of image segments. In such embodiments, a rough set based algorithm may be applied to data obtained from the anterior-posterior view scan image and the lateral view scan images to facilitate creating the plurality of image segments. The joint anatomical model may then be created based on the plurality of image segments. In another example, a user may manually select a point, portion or area of the anterior-posterior view scan image and the lateral view scan image, wherein such point, portion or area is then utilized to create the joint anatomical model. Alternatively, in some embodiments, the model may be created using the landmarks detected at 102 described above.

In some embodiments, shape and appearance information from each of the AP scout scan and the LAT scout scan may be utilized to create the model. In such embodiments, a learning based approach, for example a joint hierarchical active appearance model (AAM) may be utilized, wherein the model learns both relative positions between different parts of the landmarks and expected textures within a region of interest. The inventors have observed that by incorporating shape and appearance information with the below described AAM approach, accurate results may be produced, even in instances of substantial image noise and large structural variation.

In addition, in some embodiments, a hierarchical pyramid is employed to provide flexible incremental sub-models to reduce instances of overfitting by learning variations that occur in a single view (e.g., AP or LAT view). For example, at the first level of the hierarchical pyramid, a single joint model may be created using all of the landmarks of the manually-labelled radiographs of AP scout scan and LAT scout scan views. Such a joint model may capture the probabilistic correlation between structures in both views, which may serve to infer obscured shapes from other parts and is less sensitive to initialization errors. In subsequent finer levels of the hierarchical pyramid, sub-models are trained using scout specific vertices from the joint model, thereby allowing a more accurate and refined definition of local anatomical structures.

In an exemplary application of the AAM described above, in some embodiments, triangulated meshes based on manually or automatically annotated landmarks may be constructed to provide general locations for anatomical objects to form an initial point distribution model 200 (e.g., locations for right lung 296, left lung 204, and lung cavity 208 shown in FIG. 2A). Each area within the triangulated meshes may be a region of interest (ROI). A mean shape (shown in FIG. 2B) of the model 200 may be obtained via application of, for example, a principle component analysis (PCA) eigenanalysis. Model appearance information of each ROI may be created using, for example, an affine transformation (mean shape of each ROI shown in FIG. 2C). Subsequent models (e.g., sub-model shown in 2D) may be created in a similar manner as described above.

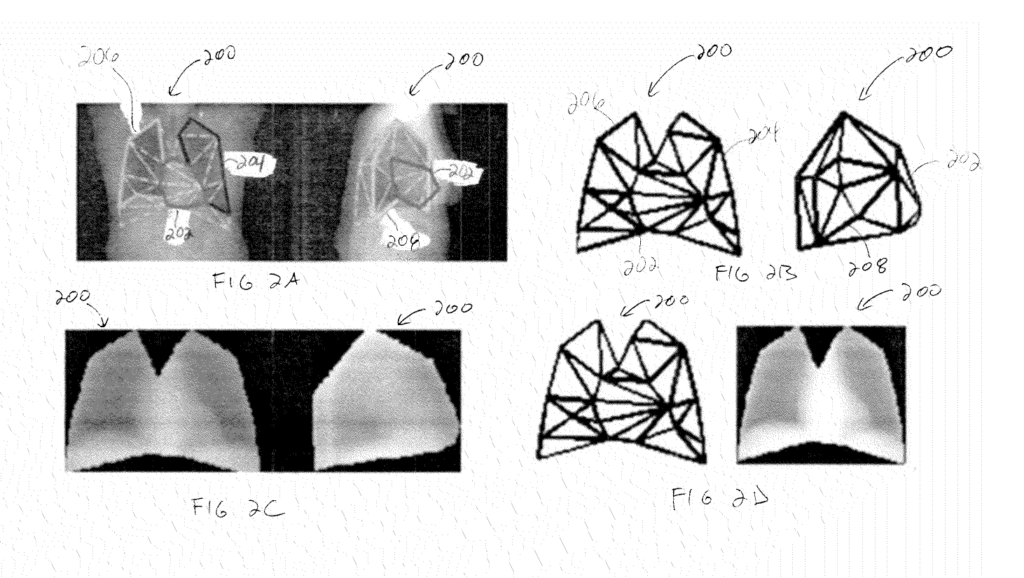

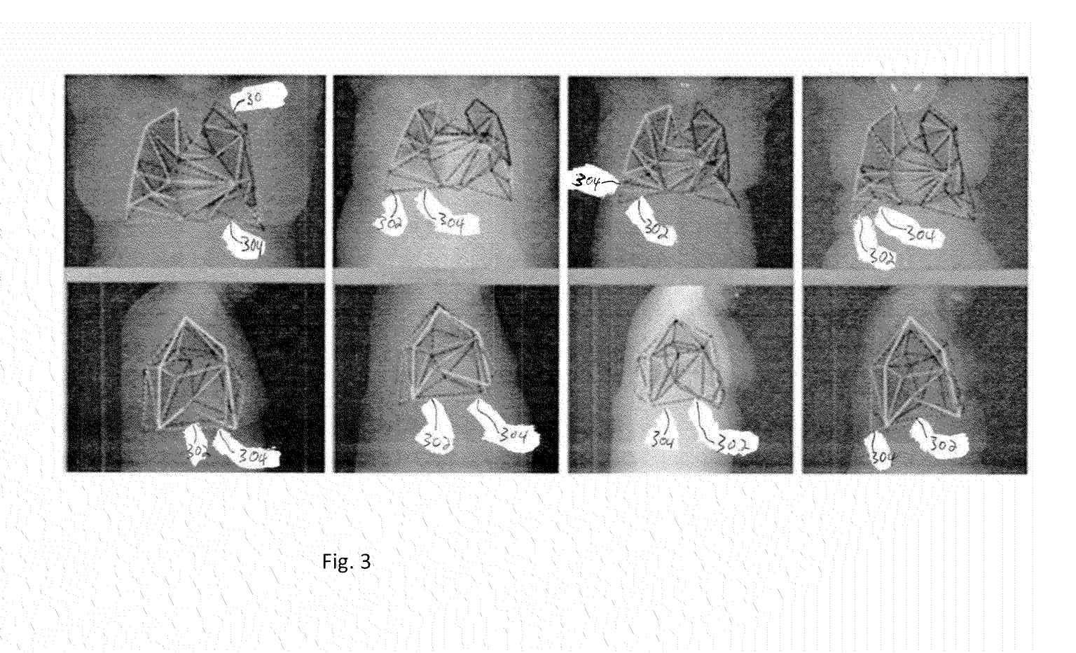

Next, at 110, the model is refined (fitted) to localize organs in both the AP and LAT views. For example, initial localization of target organs obtained via manually or automatically obtained landmarks (shown at 304) and the subsequently refined model of the target organs (shown at 304) are shown in FIG. 3

In some embodiments, the model may be refined utilizing a hierarchal approach. For example, in some embodiments, a model incorporating features from both the AP scout scan and the LAT scout scan (e.g., model 200 described above) may be fitted by minimizing a difference between the current appearance (e.g., appearance of the model) and a target image using Simultaneous Inverse Compositional (SIC) optimization. Next, localization results from the AP scout scan image may be refined by applying a sub-model learned from previously obtained AP images. The sub-model is initialized by previous joint model fitting results (e.g., sub-model creation described above) and refined using SIC. In some embodiments, to further refine LAT locations, a joint model may again be fit using SIC while keeping fixed AP landmarks. Such fixed points function as reliable anchor points, enforcing contextual constraints of LAT landmark refinement.

After the model is refined at 110, the method generally ends and the images may proceed for further processing and/or analysis. For example, in some embodiments, a bounding box may be computed using one or more landmarks along a boundary of a target organ (e.g., heart, lungs, or the like).

FIGS. 4 and 5 depicts an exemplary computed tomography (CT) imaging system 410 suitable to perform at least a portion of the method 100 described above. The CT imaging system 410 is shown as including a gantry 412 representative of a “third generation” CT scanner. Gantry 412 has an x-ray source 414 that projects a beam of x-rays 516 through a collimator assembly 411 and toward a detector assembly 418 on the opposite side of the gantry 412. Collimator assembly 411 is illustrated as a post-patient collimator that is positioned, when imaging, between a medical patient 422 and detector assembly 418. Detector assembly 418 is formed by a plurality of detectors 520 and data acquisition systems (DAS) 432. The plurality of detectors 520 sense the projected x-rays 516 that pass through medical patient 422 and are collimated by collimator assembly 411. DAS 432 converts the data from detectors 520 to digital signals for subsequent processing. Each detector 520 produces an analog electrical signal that represents the intensity of an impinging x-ray beam and hence the attenuated beam as it passes through the patient 422. During a scan to acquire x-ray projection data, gantry 412 and the components mounted thereon rotate about a center of rotation 524.

Rotation of gantry 412 and the operation of x-ray source 414 are governed by a control mechanism 526 of CT system 410. Control mechanism 526 includes an x-ray controller 528 that provides power and timing signals to an x-ray source 414 and a gantry motor controller 530 that controls the rotational speed and position of gantry 412. An image reconstructor 534 receives sampled and digitized x-ray data from DAS 432 and performs high speed reconstruction. The reconstructed image is applied as an input to a computer 536 which stores the image in a mass storage device 538.

The computer 536 may be one of any form of general-purpose computer processor that can be used in an industrial setting for controlling various systems and sub-processors. In some embodiments, the computer 536 may include a memory, CPU and support circuits. The memory, or computer-readable medium, of the CPU may be one or more of readily available memory such as random access memory (RAM), read only memory (ROM), floppy disk, hard disk, or any other form of digital storage, local or remote. The support circuits are coupled to the CPU for supporting the processor in a conventional manner. These circuits include cache, power supplies, clock circuits, input/output circuitry and subsystems, and the like. The inventive method described herein is generally stored in the memory as a software routine. The software routine may also be stored and/or executed by a second CPU (not shown) that is remotely located from the hardware being controlled by the CPU. Computer 536 also receives commands and scanning parameters from an operator via console 540 that has some form of operator interface, such as a keyboard, mouse, voice activated controller, or any other suitable input apparatus. An associated display 542 allows the operator to observe the reconstructed image and other data from computer 536. The operator supplied commands and parameters are used by computer 536 to provide control signals and information to DAS 432, x-ray controller 528 and gantry motor controller 530. In addition, computer 536 operates a table motor controller 544 which controls a motorized table 446 to position patient 422 and gantry 412. Particularly, table 446 moves patients 422 through a gantry opening 448 of FIG. 1 in whole or in part.

As commonly understood in the art, patient 422 is generally translated along a z-direction 421, or slice-direction, of gantry 412. As also commonly understood in the art, detector assembly 418 is caused to rotate circumferentially in an x-direction 423, or channel direction, of gantry 412. Thus, x-rays 516 travel generally in a y-direction 425, through collimator 411, and through detector assembly 418, as they emit from x-ray source 414 and pass through patient 422.

Thus, embodiments of a method for localizing organs in anatomical imaging have been provided. In at least one embodiment, the inventive method advantageously provides an increased localization accuracy in anatomical scans, thereby allowing for a shorter and more accurately targeted full dose scan and, thus reducing radiation dosing of a patient.

Ranges disclosed herein are inclusive and combinable. “Combination” is inclusive of blends, mixtures, alloys, reaction products, and the like. Furthermore, the terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the state value and has the meaning dictated by context, (e.g., includes the degree of error associated with measurement of the particular quantity). The suffix “(s)” as used herein is intended to include both the singular and the plural of the term that it modifies, thereby including one or more of that term (e.g., the colorant(s) includes one or more colorants). Reference throughout the specification to “one embodiment”, “some embodiments”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof Therefore, it is intended that the invention not be limited to the particular embodiment disclosed as the best mode contemplated for carrying out this invention, but that the invention will include all embodiments falling within the scope of the appended claims.

1. A method for localizing organs in anatomical imaging, comprising:
performing an anterior-posterior view scan and a lateral view scan to create an anterior-posterior view scan image and a lateral view scan image;
creating a joint anatomical model based on the anterior-posterior view scan image and the lateral view scan image; and
refining the joint anatomical model.

2. The method of claim 1, further comprising:
detecting landmarks in the anterior-posterior view scan image and the lateral view scan image; and
creating the joint anatomical model utilizing the detected landmarks.

3. The method of claim 2, wherein detecting landmarks further comprises:
creating candidate landmark locations from the anterior-posterior view scan image and the lateral view scan image; and
correcting false positives and false negatives from the candidate landmark locations.

4. The method of claim 1, further comprising:
segmenting the anterior-posterior view scan image and the lateral view scan image to provide a plurality of image segments; and
creating the joint anatomical model based on the plurality of image segments.

5. The method of claim 4, wherein segmenting the anterior-posterior view scan image and the lateral view scan image comprises applying a rough set based algorithm to data from the anterior-posterior view scan image and the lateral view scan image.

6. The method of claim 1, further comprising:
manually selecting a portion of the anterior-posterior view scan image and a lateral view scan image; and
creating the joint anatomical model based on the manually selected portion.

7. The method of claim 1, wherein creating the joint anatomical model comprises:
utilizing a joint hierarchical active appearance model, wherein the joint hierarchical active appearance model learns relative positions of portions of the anterior-posterior view scan image and the lateral view scan image and expected textures within a region of interest.

8. The method of claim 1, wherein refining the joint anatomical model comprises:
minimizing a difference between a current appearance of the joint anatomical model and a target image using Simultaneous Inverse Compositional (SIC) optimization.

9. A computer readable medium, having instructions stored thereon which, when executed, causes an imaging system to perform a method for localizing organs in anatomical imaging, the method comprising:
performing an anterior-posterior view scan and a lateral view scan to create an anterior-posterior view scan image and a lateral view scan image;
creating a joint anatomical model based on the anterior-posterior view scan image and the lateral view scan image; and
refining the joint anatomical model.

10. The computer readable medium of claim 9, further comprising:
detecting landmarks in the anterior-posterior view scan image and the lateral view scan image; and
creating the joint anatomical model utilizing the detected landmarks.

11. The computer readable medium of claim 10, wherein detecting landmarks further comprises:
creating candidate landmark locations from the anterior-posterior view scan image and the lateral view scan image; and
correcting false positives and false negatives from the candidate landmark locations.

12. The computer readable medium of claim 9, further comprising:
segmenting the anterior-posterior view scan image and the lateral view scan image to provide a plurality of image segments; and
creating the joint anatomical model based on the plurality of image segments.

13. The computer readable medium of claim 12, wherein segmenting the anterior-posterior view scan image and the lateral view scan image comprises applying a rough set based algorithm to data from the anterior-posterior view scan image and the lateral view scan image.

14. The computer readable medium of claim 9, further comprising:
manually selecting a portion of the anterior-posterior view scan image and a lateral view scan image; and
creating the joint anatomical model based on the manually selected portion.

15. The computer readable medium of claim 9, wherein creating the joint anatomical model comprises:
utilizing a joint hierarchical active appearance model, wherein the joint hierarchical active appearance model learns relative positions of portions of the anterior-posterior view scan image and the lateral view scan image and expected textures within a region of interest.

16. The computer readable medium of claim 9, wherein refining the joint anatomical model comprises:
minimizing a difference between a current appearance of the joint anatomical model and a target image using Simultaneous Inverse Compositional (SIC) optimization.
